(19) United States
(12) Patent Application Publication
(10) Pub. No.: US 2014/0073905 A1
(43) Pub. Date: Mar. 13, 2014

(54) SYSTEMS AND METHODS FOR BLOOD OXYGEN LEVEL DEPENDENT MRI

(71) Applicant: Advanced Neuro Therapeutics, LLC, Santa Monica, CA (US)

(72) Inventors: Sheldon Jordan, Santa Monica, CA (US); Bradley A. Jabour, Santa Monica, CA (US)

(73) Assignee: Advanced Neuro Therapeutics, LLC, Santa Monica, CA (US)

(21) Appl. No.: 14/025,774

(22) Filed: Sep. 12, 2013

Related U.S. Application Data
(60) Provisional application No. 61/700,297, filed on Sep. 12, 2012.

(51) Int. Cl. A61B 5/00; A61B 5/055

(52) U.S. Cl. CPC A61B 5/0042; A61B 5/055
USPC 600/410

(57) ABSTRACT

Abnormal functioning of the dorsal lateral prefrontal cortex (DLPF) has been implicated in depression. Repetitive Transcranial Magnetic Stimulation (rTMS) of the DLPF has been successful in treating depression, however, successful translation to routine clinical practice has shown modest results using standard protocols. The present invention provides paradigms, systems, and methods for the targeted, location specific, and pulse-modulated treatment of conditions such as depression, anxiety, OCD, chronic pain syndromes, drug and alcohol addiction, and other conditions through the use of advanced functional MRI (fMRI) or PET/CT, stereotactic neuronavigation, and the performance of cognitive tasks with the maximally efficient delivery of rTMS pulses, which can be varied and precisely targeted, to obtain concurrent activation of targeted brain networks.

### FIELD OF THE INVENTION

The present disclosure relates generally to functional magnetic resonance imaging (fMRI) and neuronavigation imaging techniques and equipment for guiding repetitive Transcranial Magnetic Stimulation (rTMS).

### BACKGROUND

Depression is the leading brain disease affecting hundreds of millions of individuals around the world, and an estimated 1 in 10 individuals in the US. Pharmacological and behavioral treatments are not always effective and electroconvulsive therapy (ECT) is not always preferred by patients. Alternative modalities have become available including repetitive Transcranial Magnetic Stimulation (rTMS) which has recently been approved by the Food and Drug Administration (FDA). rTMS for the treatment of depression has been the subject of many clinical trials and meta-analytic studies; however, this treatment modality has not always produced large treatment effects or consistently positive outcomes. For example, a recent randomized, controlled, multi-institutional study of rTMS to the left dorsal lateral prefrontal area demonstrated only a 23.9 percent frequency of 50% improvement in depression scale scores compared to a 12.3 percent rate in sham control patients. Furthermore, it is not entirely clear from published studies how well any potential benefits may translate to clinical practice where medications cannot always be withheld as they have often been in experimental series. Furthermore, it is not clear from the available literature how well non-dextrals fare with left hemisphere targeting as compared with right handers.

Imaging based neuronavigation has been suggested to establish more reliable and effective magnet placements. Since the inception of rTMS therapy the focus of targeting efforts has been the dorsal lateral prefrontal region because clinical depression been observed after injury to this region and because anatomical studies of the dorsal lateral prefrontal area demonstrate connections to limbic regions that are proposed to be implicated in the pathophysiology of depression. The finding of hypometabolism with PET scans in patients with depression has prompted several studies using PET data for the purposes of imaging guided neuronavigation. No improved outcomes have been demonstrated when the most hypometabolic hemisphere was targeted based on PET data. It is important to note that each of the reported studies utilized very brief treatment trials and there have been no prolonged attempts to target hypometabolic regions within the chosen (typically left) hemisphere. More recent efforts have utilized fMRI imaging data sets including those obtained with connectivity analysis of resting state BOLD for the purposes of navigation with the concept that the latter approach may supersede PET based techniques.

The advent of ASL and fMRI utilizing blood oxygenation level dependent fMRI techniques has allowed imaging specialists to identify the abnormal network foot print in the brain in patients with depression, obsessive-compulsive disorders, anxiety, CRPS, drug and alcohol dependency, and severe pain syndrome. rTMS has been extensively studied in the treatment of depression and has been FDA approved for a number of years. There is a need for a process or method to locate, to quantify, and then to treat a number of neuropsychiatric, behavioral, neurological, and pain disorders (amongst others) using fMRI, a stereotactic navigation system, a novel method of biomarker quantification, and rTMS.

### BRIEF SUMMARY

In preferred embodiments of the invention, methods and systems are provided for utilizing a variety of digital imaging modalities, including but not restricted to Positron emission tomography (PET)/computed tomography (CT), magnetic resonance imaging (MRI), and/or functional MRI (fMRI) examinations to obtain anatomic and functional images of the brain using a variety of sequences and techniques in each of the modalities. In addition, the technique may include use of fludeoxyglucose (FDG) PET scans as well as other radionuclides. Utilizing blood oxygenation level dependent (BOLD) techniques and fMRI as well as resting state BOLD (also known as state dependent BOLD), and provocative stimulated BOLD examination and new techniques such as arterial spin labeling (ASL). During the acquisition of the images, a number of techniques have been developed by this team to enhance functional brain data, amongst which are EAST (elevated arm stress test), LEST (leg elevated stress test), GIST (guided imagery stimulation test) and the pressure stimulation study (PSS). The digital data obtained from these images are further analyzed using independent component analysis software to further enhance and display both anatomic and functional detail. Using Prepared Rapid Acquisition Gradient Echo (MPRAGE) and/or Spoiled Gradient Recalled Acquisition (SPGR) sequences as an anatomical foundation, the functional data set is then superimposed upon it. As a template, the functional data digital sets are analyzed and displayed using independent component analysis and are then utilized on a stereotactic neurosurgical device (e.g. BrainLAB Kolibri) in order to highlight the patient anatomy and functional data. A transcranial magnetic stimulation device is then more accurately targeted to the specific portion of the brain using these neuronavigation techniques.

This brief summary is provided to introduce a selection of concepts in a simplified form that are further described in the detailed description. It is not intended to be exhaustive or to limit the inventions to the precise forms disclosed.

### BRIEF DESCRIPTION OF THE DRAWINGS

Preferred embodiments of the present invention are illustrated by way of example, and not by way of limitation, in the figures of the accompanying drawings and in which like reference numerals refer to similar elements and in which:

FIG. 1 is an exemplary illustration of an imaging system, such as an MRI, in which a patient receives a brain scan according to aspects of some embodiments of the invention.

FIG. 2 is an exemplary illustration of a computer and method for conducting a brain scan on a patient according to aspects of some embodiments of the invention.

FIG. 3 is an exemplary illustration of a computer and associated equipment for delivering rTMS pulses after the computer has captured an fMRI brain scan of a patient while undergoing various baseline and stress tests in an fMRI machine, in accordance with some aspects of a preferred embodiment of the invention.

FIG. 4 is an exemplary illustration of how a computer, associated equipment for delivering rTMS pulses after the computer has captured an fMRI brain scan of a patient while undergoing various baseline and stress tests in an fMRI machine, and the patient who underwent the various baseline and stress tests in the fMRI machine, function together to deliver rTMS pulses to targeted areas in the patient's brain, in accordance with some aspects of a preferred embodiment of the invention.

FIG. 5 is an exemplary illustration of a patient has undergone various baseline and stress tests in an fMRI machine and who has been marked externally on his head to represent the area of his brain to which to target the delivery of rTMS pulses to treat various conditions, in accordance with some aspects of a preferred embodiment of the invention.

FIG. 6 is an exemplary illustration of a patient, who underwent various baseline and stress tests in an fMRI machine and who's head has been physically marked to indicate to what areas in the patient's brain rTMS pulses should be delivered, receiving rTMS pulses from a system comprising a computer that captured the fMRI brain scan of the patient while he was undergoing various baseline and stress tests in the fMRI machine and the machine that delivers rTMS pulses itself, in accordance with some aspects of a preferred embodiment of the invention.

FIG. 7 is an exemplary system diagram depicting the use of a computer system for implementing aspects of preferred embodiments of the invention.

FIG. 8 is an exemplary drawing of a sample report for an OCD patient following rTMS with significant treatment effect.

FIG. 9 is an exemplary drawing of a sample report for an OCD patient following rTMS with significant treatment effect.

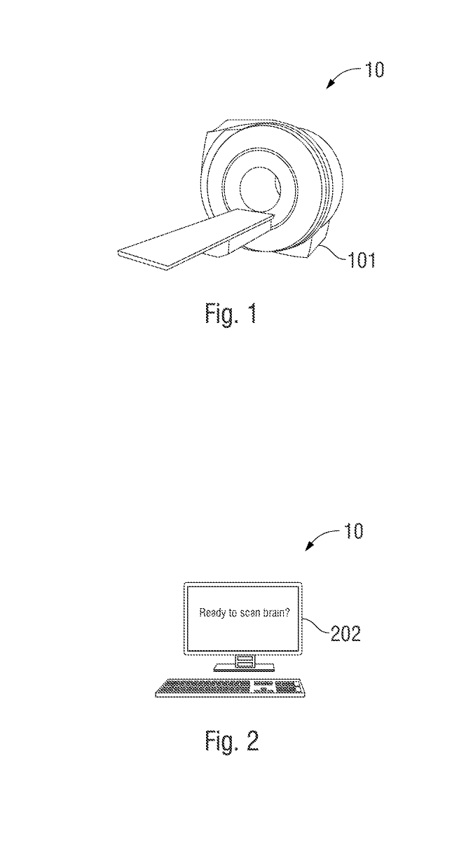

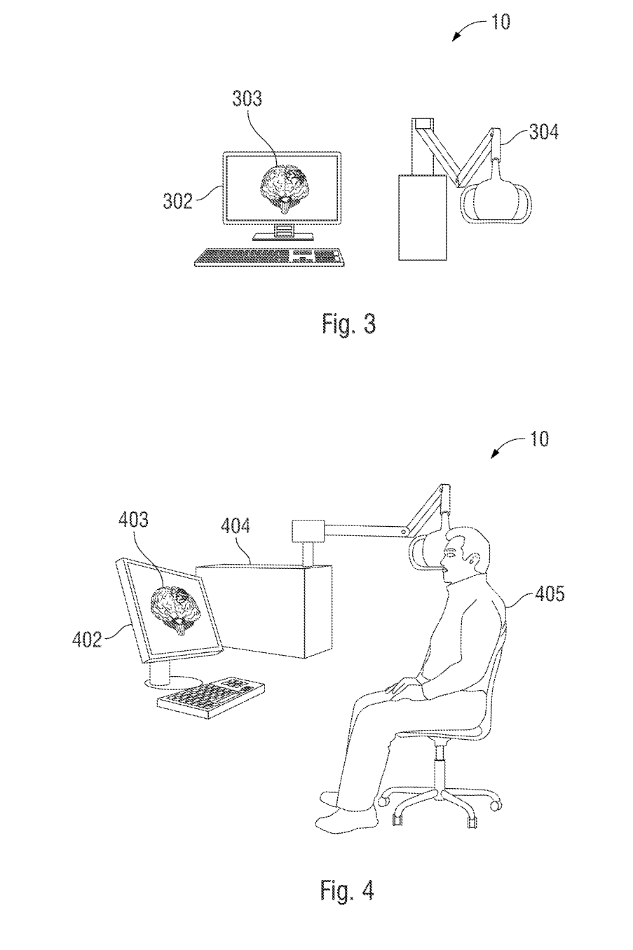

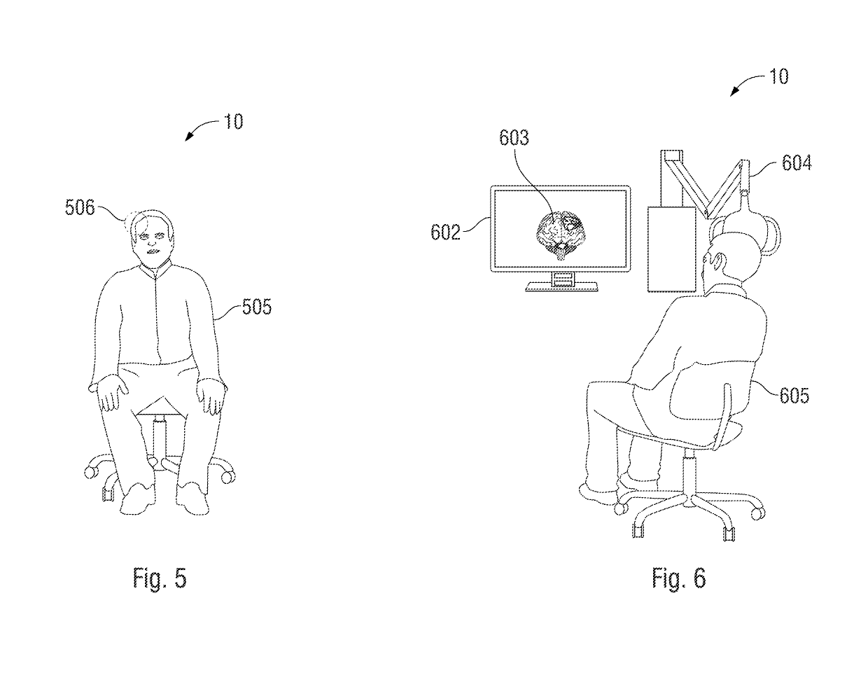

### DETAILED DESCRIPTION OF PREFERRED EMBODIMENTS OF THE INVENTION

Other and further features and advantages of the present invention will be apparent from the following descriptions of the various embodiments when read in conjunction with the accompanying drawings. It will be understood by one of ordinary skill in the art that the following embodiments are provided for illustrative and exemplary purposes only and that numerous combinations of the elements of the various embodiments of the present invention are possible.

The present invention operates to image the neurosensory cortex and the cortical representation of pain using BOLD MRI sequence stimulated by increasing pain levels with elevated arm stress tests (EAST), leg elevated stress tests (LEST), and pressure stimulation studies (PSS) as follows.

The BOLD MRI sequence is performed with the brain in a resting state, or in a sequence run while the patient is exposed to different pain stimuli.

The first stimulus, is a pressure cuff applied to the forearm or leg which is pumped up to 300 mmHg or to a level consistent with a patient's perception of pain, whichever is lower, for periods of less than one minute as the patient is being scanned in the MRI scanner. MRI signal obtained with BOLD or ASL techniques after exposure to resting and stimulated states are compared.

The second stimulus is the Elevated Arm Stress Test (EAST) during which the patient elevates the tested arm and opens and closes the first for thirty to sixty seconds.

The third stimulus, the Leg Elevated Stress Test (LEST), is performed by having the patient lift a lower extremity up to forty five degrees and then internally and externally rotate the limb with the knee straight for thirty to sixty seconds. For both the FAST and LEST, MRI data is obtained with BOLD or ASL pulse sequences in the resting and activated states, and the two are compared.

The invention may also use BOLD MRI with independent component analysis to determine targets for repetitive transcranial magnetic stimulation of the orbital frontal area as follows.

After use of the above technique, one pattern that is identified with both Resting and State Dependent (Stimulated) conditions is a pattern related to the Orbital Frontal Region which is often seen as a part of the so-called “Default Network”. The default network is identified by increased signal prominence in the precuneus area. We have perfected a means of treating obsessive compulsive disorder by targeting the medial orbital frontal region with rTMS using slow rates of stimulation (less than five per second, typically at 1 hertz).

BOLD MRI may be used with ICA to determine the location of the orbital frontal regions or the frontal executive regions in a patient for the determination of targeting for Repetitive Transcranial Magnetic Stimulation (rTMS). The MRI data set is transferred to a neuronavigation device that co-registers the MRI data set (and any imaging data sets with which it may be merged) with a person's actual brain. Areas of MRI signal related to the orbital frontal areas or frontal executive areas may then be identified and with the use of the navigation device, used to accurately target the rTMS magnetic-pulse to the target brain anatomy.

The invention may also use BOLD MRI with independent component analysis to determine targets for repetitive transcranial magnetic stimulation of the frontal executive areas as follows.

Using the BOLD technique either in resting or state dependent conditions, it is possible to identify a network that involves the bilateral frontal regions in a more or less symmetrical fashion which has variously been called the frontal executive network and which also goes by other identifiers. This identified region can then be used to target the transcranial magnetic stimulations for the purposes of treating a variety of conditions including but not limited to depression and a variety of pain states.

The invention may also use the Guided Imagery Stimulation Task (GIST) to perform its methods and systems for treatment as follows. GIST is performed by having the fMRI patient view a visual display on a screen that presents three words arranged with one word in each of three lines. The lines are arranged centered on the display with a gray background. Each sample of three words is displayed for thirty seconds and then replaced with another set of three words. 10 sets of 3 words are available for display out of a sample of 30 words. The words are developed upon direct interview with the patient in each case.

The BOLD sequence is run identically (same scan parameters) for both resting state and with GIST activation.] Thus, the activation of the executive area of the brain by having the patient focus on the word triplets during the fMRI scan is the difference between the two BOLD sequences.

With each thirty second long epoch, the patient is instructed to pick one of the three words and, using all five senses, imagine a scene that is evoked by the word in which they are themselves participating in a healthy, pleasurable, energetic, and productive fashion. When they feel they have been successful in imagining the scene, then they will press an event recorder button. With subsequent three word displays, the patient will repeat the same process. However, when a particular three word set reappears the patient is instructed to choose a different word to evoke an imagined scene.

The thirty word set is developed with the patient in advance by asking each person to identify words that represent actual people, places and activities that they would want to experience good times with in the future when they are feeling healthy.

The invention also allows for the use of arterial spin labeling to determine targets for repetitive transcranial magnetic stimulation as follows.

Arterial spin labeling (ASL) is an MRI based imaging technique that allows for imaging of the brain with signal that is partly proportional the flow of blood into brain tissues. The resultant imaging data sets can be visualized alone to show brain perfusion and or metabolism (analogous to PET/FDG scan). These ASL images can be fused with MPRAGE or SPGR with other sequences to provide better anatomical detail.

ASL can be used in the determination of targeting for rTMS. The ASL data set is to be transferred to a neuronavigation device that co-registers the ASL data set (and any imaging data sets with which it may be merged) with a person's actual brain. Areas of increased or decreased ASL signal may then be identified and with the use of the navigation device, used to accurately target the rTMS magnetic pulse to the assigned anatomic location.

The invention also provides for systems and methods to targeting the ventral lateral/lateral orbital prefrontal area for treating pain as follows.

In the past, several targets have been used for treating pain including the sensorimotor cortex and the dorsal lateral prefrontal region. A ventral lateral/lateral orbital target has been developed for treating pain with either faster (such as ten Hertz rate) or slower (such as one Hertz rate) stimulation. This area has been shown to be relevant for the Placebo response and is, therefore, another target area that can be used in the treatment of pain.

The following is a list of manufacturer descriptions and acronym explanations for the various machines, equipment, and tests that can be used to perform the inventive methods and to comprise the inventive systems described herein at the time of the invention. Such later developments may be substituted to achieve the claimed invention.

MACHINES. MRI Machines: Siemens, GE, and Phillips. Stereotactic Neuronavigation: Brain Lab, Medronic, rTMS, Neuronetics, Brainsway. Sensory Stimulation: Medoc. fMRI: Siemens.

ACRONYMS: MRI stands for magnetic resonance imaging. fMRI stands for functional magnetic resonance imaging. PET stands for positron emission tomography. CT stands for computed tomography. ASL stands for Arterial Spin Labeling. GIST stands for Guided Imagery Stimulation Task. EAST stands for Elevated Arm Stress Test. LEST stands for leg elevated stress test. PSS stands for pressure stimulation study. ECT stands for electroconvulsive therapy. MPRAGE stands for Magnetization Prepared Rapid Acquisition Gradient Echo. SPGR stands for Spoiled Gradient Recalled Acquisition. CRPS stands for complex regional pain syndrome. FDG stands for fludeoxyglucose. DLPF stands for dorsolateral prefrontal cortex. BOLD stands for blood oxygenation level dependent. rTMS stands for repetitive Transcranial Magnetic Stimulation.

In preferred embodiments of the invention, the inventive process involves the novel use of a number of independent technologies including 3T, MRI, Stereotactic Neuronavigation Systems, fMRI, ASL, rTMS, and a System of Biomarkers to enable the accurate Neuronavigation of the brain and its networks and structures, to identify abnormalities (using a metric and system of biomarkers) and to measure the brain's responses to rTMS for a variety of neuropsychiatric, behavioral response, neurological, and pain disorders.

The following issues and conditions may be treated by the inventive systems and methods described herein.

Depression. This invention utilizes a process whereby in the setting of depression, hypometabolic dorsolateral prefrontal cortex (DLPF) is located, the abnormality quantified the level of connectivity in the executive center and in the pretreatment phase. The Biomarkers developed by this team allow us to follow progress during and post treatment. Depression is treated following the utilization of the neuronavigation techniques using both fMRI, ASL and the stereotactic Brain Lab by using localization followed by rTMS treatment at 10 hertz. The patient's response is measured with analysis of subsequent fMRI studies in the mid treatment and at the end of treatment confirming the patient's response. In addition, traditional measurements are followed by Yale Brown, Beck, etc.

Obsessive-Compulsive Disorder likewise has a particular foot print as evidenced on the fMRI, whereby the BOLD activity in the medial frontal lobe in the orbitofrontal cortex has a particular pattern. This allows the abnormality to be measured with the biomarker techniques (developed by our team) pretreatment and then the area is targeted and 1 hertz of rTMS is used to suppress the aberrant activity. The patient is also followed with fMRI studies mid treatment and post treatment confirmed response. At multiple intervals in treatment cycles, the Yale Brown neurocognitive tests are performed to evaluate the OCD criteria clinically.

Anxiety syndromes are treated by targeting the right dorsolateral prefrontal cortex with 1 hertz (in right-handed individuals).

Chronic pain syndromes such as complex regional pain syndrome (CRPS), fibromyalgia are targeted by using suppressive strategies of 1 hertz to the medial frontal cortex.

Patients with dependency disorders (alcohol and/or drugs) are also treated by targeting the orbitofrontal cortex, medial frontal lobe.

The following traditional clinical selection methods are used to select patients for receipt of the novel treatment described herein:

Patients with Depression. Patients with typical symptoms of depression who have been treated with anti-depressant medications with unsatisfactory response are selected for treatment with RTMS. Once identified as being appropriate, the patient undergoes an MRI study to evaluate brain morphology and an fMRI study using both baseline BOLD and arterial spin labeling. In addition, an fMRI and ASL study are performed to engage the executive brain unit GIST techniques developed by our team. Using the Biomarkers developed from our proprietary normative data base, the patient's resting state network is compared with similar age-related data sets whereby the default mode network and executive areas of the brain together with the arterial spin labeling images to measure activity and blood flow/metabolism in the dorsolateral prefrontal cortex. Once the imaging confirms the appropriate hypometabolic area, the patient's 3-dimensional data sets (MPRAGE) are transferred onto a stereotactic neuronavigation system used in neurosurgery (made by Brain Lab or Medronic) and then the appropriate area of the brain is demarcated in a neuronavigation session to direct. This area is treated with rTMS at 10 hertz in the case of depression. We have also developed a guided imagery protocol to better identify the executive area during the fMRI study, and to utilize during treatment with rTMS to enhance the brain response.

Patients with OCD: Similarly, in OCD, the abnormal area, in this case, the medial frontal cortex of the orbitofrontal brain is identified with the fMRI and neuronavigational techniques and is then suppressed with rTMS often bilaterally. Again, Biomarkers and GIST techniques are used to evaluate response and to enhance treatment of the brain at various points in the treatment cycle.

Patients with Anxiety. The fMRI will often reveal increased activity on the resting state network default mode in the amygdala. This together with the patient's clinical symptoms will indicate to our team that the right dorsolateral prefrontal cortex could be suppressed with 1 M hertz.

Patients with Chronic Pain Syndromes. Patients with reflex sympathetic dystrophy, also known as chronic regional pain syndrome, will have particular finding on the resting state networks, in particular, in the salience network. The medial orbitofrontal cortex is targeted and the salience network and the fMRI techniques are used to follow the response with our Biomarker fMRI system.

Patients with Drug Dependency and Addiction. The orbitofrontal cortex is also targeted in a similar fashion to OCD.

In a preferred embodiment, the following systems and methods will be followed to treat a patient in accordance with aspects of the claimed invention 10. A patient is evaluated through an initial pre-consultation history to determine the patient's qualification for rTMS treatment. If a patient is deemed to potentially qualify, they are referred for a functional MRI (fMRI) study.

The fMRI study will act as a baseline of information on patients actually admitted into the program. Unique paradigms are used in concert with a free Tesla MRI system 101. MRI systems themselves are capable of assisting in the performance of various paradigms, but these paradigms have been custom designed by the inventors. The baseline examination will demonstrate the specific area of the brain for targeting the rTMS magnetic pulses.

Images acquired 303 403 603 on the 3T fMRI system 101 are post processed using a number of devices including an Invivo Workstation 202 302 402 602, a Medoc Workstation 202 302 402 602, and a Brain Lab navigation device 304 404 604.

The Brain Lab device 304 404 604 is used for surgical intervention. This unique process for using the Brain Lab device in a non-interventional manner allows for mapping of the real-time brain such that targeting of the rTMS pulse can be highly specific.

The Brain Lab device 304 404 604 houses the image of the brain 303 403 603, which shows the area of hypo or hypermetabolic activity to be stimulated or repressed. This image is used with the actual patient present. A technologist uses a biometric device to map the real-time brain, correlating the area mapped to the imaged brain appearing on the device. This allows for a specific marker 506 to be placed at the area on the patient 405 505 605 where the rTMS is to be specifically targeted.

The patient 405 505 605 returns at regular intervals for treatment and has a specific marker 506 so that each time a treatment is performed, the area to be targeted can be specifically identified by the technologist performing the treatment.

The inventors use the Neuronetics Neuro Star unit. This unit was not designed with neuronavigation in mind. Instead, it was designed such that a technologist would target a general 5 cm area from motor cortex. As a result, the traditional treatments have less efficacy. By using neuronavigation with the Neuro Star device, the rTMS unit can be specifically placed to target the specific area of the brain which requires the greatest focused targeting. During the course of treatment, certain fMRI paradigms are repeated to determine the patient's progress on an objective imaging study. At the end of treatment, a further MRI study is performed that can show the difference between the baseline image and the post treatment image and the resultant improvement. These quantitative techniques using fMRI biomarkers have been developed by our team.

The following study conducted using the inventive systems and methods described herein demonstrates preferred aspects of preferred embodiments of the invention. This study and all the references to which it cites are fully incorporated herein by reference.

Title.

Treatment of Depression with Repetitive Transcranial Magnetic Stimulation Targeted at Functionally Mapped Prefrontal Cortex with Concurrent Cognitive Task Performance: Translation to Clinical Practice, A Post Hoc Analysis.

Background.

Abnormal functioning of the dorsal lateral prefrontal cortex (DLPF) has been implicated in depression. Repetitive Transcranial Magnetic Stimulation of the DLPF has been successful in treating depression, however, successful translation to routine clinical practice has shown modest results using standard protocols. We have shown dramatic improvements in patient response with the use of advanced functional MRI (fMRI) or PET/CT, stereotactic neuronavigation and the performance of cognitive tasks with rTMS to obtain concurrent activation of targeted brain networks.

Methods.

Eighty two patients with moderate to severe depression were treated. Initially, twelve patients were treated using the manufacturer supplied head holder measurement device and targeted by placing the magnetic coil 5 cm anterior to the identified motor area. Subsequently, the DLPF area was targeted in seventy three patients using an imaging based neuronavigation system with PET/CT and/or fMRI data sets. Outcomes were compared in those patients having imaging guided neuronavigation with those that were targeted without imaging guidance. In addition, patients performed a guided imagery task during treatment sessions.

Results.

Remission was achieved in forty seven of 73 patients (66%) with imaging based neuronavigation and a concurrent cognitive task compared to 3 of 12 patients (25%) in the non neuronavigated group (p=0.0109 Fisher Exact). Imaging based navigation facilitated rapid targeting of the motor area and enabled more reliable targeting of pre-frontal treatment sites.

Conclusions.

Combining imaging based neuronavigation for targeting rTMS with the performance of a concurrent cognitive task during treatment sessions often extending past four weeks appears to improve patient outcomes in treating depression.

Introduction.

Depression is the leading brain disease affecting hundreds of millions of individuals around the world, and an estimated 1 in 10 individuals in the US (ref 1). Pharmacological and behavioral treatments are not always effective and ECT is not always preferred by patients. Alternative modalities have become available including repetitive transcranial magnetic stimulation (rTMS) which has recently been approved by the FDA. Repetitive transcranial magnetic stimulation (rTMS) for the treatment of depression has been the subject of many clinical trials and meta-analytic studies; however, this treatment modality has not always produced large treatment effects or consistently positive outcomes. For example, a recent randomized, controlled, multi-institutional study of rTMS to the left dorsal lateral prefrontal area demonstrated only a 23.9 percent frequency of 50% improvement in depression scale scores compared to a 12.3 percent rate in sham control patients (ref 2). Furthermore, it is not entirely clear from published studies how well any potential benefits may translate to clinical practice where medications cannot always be withheld as they have often been in experimental series. Furthermore, it is not clear from the available literature how well non-dextrals fare with left hemisphere targeting as compared with right handers.

The first phase of the present clinical experience reports on problems encountered with the translation of routine protocols utilizing a proprietary targeting device to a clinical population.

The initial phase of the clinical experience utilized standard treatment protocols as prescribed by the manufacturer. Targeting was performed using the “five centimeter rule” based on advancing the treatment magnet five centimeters anterior to a scalp site that was associated with low threshold motor activation of the contralateral hand. As will be noted, failure in quality assurance and low clinical remission rates in the application of routine targeting protocols prompted the application of improved targeting based on frameless stereotaxis with imaging based neuronavigation for the second phase of this clinical experience.

Imaging based neuronavigation has been suggested to establish more reliable and effective magnet placements (ref 3). Since the inception of rTMS therapy the focus of targeting efforts has been the dorsal lateral prefrontal region because clinical depression been observed after injury to this region and because anatomical studies of the dorsal lateral prefrontal area demonstrate connections to limbic regions that are proposed to be implicated in the pathophysiology of depression (ref 4). The finding of hypometabolism with PET scans in patients with depression (ref 4, 5, 6) has prompted several studies using PET data for the purposes of imaging guided neuronavigation. No improved outcomes have been demonstrated when the most hypometabolic hemisphere was targeted based on PET data. It is important to note that each of the reported studies utilized very brief treatment trials and there have been no prolonged attempts to target hypometabolic regions within the chosen (typically left) hemisphere. More recent efforts have utilized fMRI imaging data sets including those obtained with connectivity analysis of resting state BOLD for the purposes of navigation with the concept that the latter approach may supersede PET based techniques.

Phase two of this clinical experience reports on efforts use both PET/CT and fMRI imaging data coupled with an frameless stereotaxic system for more accurate positioning. With the hope of avoiding the radiation exposure and cost or PET/CT imaging, we expected to make a transition to the use of MRI and fMRI for targeting. Precise targeting with regards to sulcal anatomy was expected to be an important factor in improving outcomes since it has been demonstrated that activation effectiveness appears to coincide with the bank of a sulcus. A projected improvement in the five centimeter rule was, therefore, to intersect the five centimeter measurement to a point along the superior central sulcus of the dorsal lateral prefrontal target area rather than allowing it to be randomly placed with respect to the patient's sulcal anatomy. In addition, because previous fMRI studies have demonstrated activation of frontal executive networks while subjects performed tasks related to emotional tasks, it seemed appropriate to target this network as identified by independent component analysis of resting BOLD imaging data as one factor in refining the 5 centimeter rule.

In addition, patients in phase two were engaged in a guided imagery task throughout treatment sessions in order to maintain a state of alertness and to “pre-activate” the targeted dorsal lateral prefrontal lobe (DLPF) rather than allowing the patient to repeatedly drift to sleep or experience random cognitive states.

Phase two of the clinical experience with it use of imaging based neuronavigation and the use of a cognitive task to maintain patient focus was predicted to improve accuracy of targeting and improve outcomes as measured by remission rates and duration of remissions. The outcomes of left handers were to be specifically tracked as it was expected that approximately ten percent of the clinical sample would include left handers.

It was hoped that the present analysis of a clinical experience would be instructive in ways which will lead to additional controlled trials and will foster additional modifications for the successful translation of rTMS into routine psychiatric practice.

Methods.

Two subsets of patients were treated at Smart Brain and Health using rTMS with and without neuronavigation.

Subset One: rTMS Utilized with Standard Protocols (without Neuronavigation and Imaging).

Patients were treated with rTMS in this clinical series for major depression that could be treated as an outpatient over the course of six weeks and who had failed to receive satisfactory improvement from at least one prior antidepressant medication at or above the minimal effective dose and duration in the current episode. Patients with metal implants or devices within 30 cm of the target site were excluded except for patients with dental implants. Patients with minor or transient psychotic features such as auditory hallucinations were not excluded. No attempt was made to wean patients off medications prior to or during treatment courses. Patients taking benzodiazepines or other medication that might interfere with rTMS were advised to maintain constant scheduled dosing rather than using them sporadically and to avoid any dosage changes during the treatment period.

A Neuronetics system which has a coil design with a ferromagnetic core and a design which is similar to a figure of eight configuration modified to a double square. For the first phase that was performed without imaging navigation, the “five centimeter rule” was used to determine the treatment site in the left dorsal lateral prefrontal region. First, the sensorimotor area was defined by lowest threshold activation of the contralateral fingers; this area was then marked and, using the Neuronetics head holder and measurement device, the treatment target was determined 5 centimeters anterior on the scalp. Quality assurance protocols included repeated determinations of the final treatment site determined by the technician compared to a scalp positions determined by the 10-20 International Electroencephalographic System by an individual board certified by the American Board of Clinical Neurophysiology. Any intersession disparity greater than one centimeter was noted as a quality assurance failure.

Treatment was directed initially at the left dorsal lateral prefrontal area according to the 5 centimeter rule for at least 3000 stimulations per session at 120 percent of motor threshold at a stimulus rate of ten per second. For patients who did not appear to have a substantial improvement after two weeks, a second stimulus site was chosen over the right prefrontal area using the five centimeter rule and stimulation at one per second for 2000 stimulations per session added to continued left dorsal lateral stimulation at ten per second.

Patients were allowed to listen to music or sit quietly in the room with eyes open or closed. No specific behavioral intervention was utilized.

As a primary measurement for a good outcome was a fifty percent or greater change in a Zung depression scale along with a patient's self assessment of a meaningful improvement in mood and daily activities.

Subset Two: rTMS Utilizing Neuronavigation Image Guidance and Concurrent Cognitive Task.

Patient selection was the same as in the first phase. Stimulation protocols for dorsal lateral prefrontal targets were the same as well. A Brainlab Kolibri Neuronavigation System (Brainlab AG, Feldkirchen, Germany) was used for targeting the rTMS magnet. PET/CT and MRI data, transferred to the Brainlab device, allowed for motion correction, co-registration and display of the imaging data sets. The contour of the patient's real face was then co-registered by reflecting laser beams on the face which was then analyzed by the Brainlab's camera and computer system, and matched to the contour of the virtual face of the computerized image. Any landmark on the patient's real head was then co-registered to a corresponding landmark on the virtual scalp and to any brain structures underneath. A magic wand, tracked in real time by the Brainlab was then used to point out targets that were chosen in the planning stages to correspond to DLPF, and the motor strip. Care was taken to point to a structure at depth in the virtual brain that was determined with the magic wand forming a perpendicular angle to the scalp surface. The real scalp projection of the brain target was made with a permanent pen marker so that the Neuronetics coil would be centered over the mark with proper angulation determined by the coils integrated sensors. Accurate targeting is then based on an accurate scalp centering mark and the coil's angle sensors. Depth penetration of the changing magnetic field effect is proportional to the intensity of the Neuronetics system settings and is limited both by the machine's output ceiling as well as patient's tolerance of scalp stimulation. Modeling of this magnet design has suggested effective depth penetration of approximately 2 cm for producing an induced electrical field of 140 V/m that would be predicted to activate the motor strip in an average subject.

Forty two patients were targeted with a PET/CT data set, 15 were targeted with MRI and 13 had both PET/CT scans of the brain were acquired as follows. Images were acquired using a GE Discovery ST PET/CT scanner 45-60 minutes following the intravenous injection of 444-555 MBq (12-15 mCi) 18-Fluoro-2-deoxyglucose (FDG), with the patient fasting, after determinations of blood glucose concentrations. Patients remained awake and at rest in a quiet, dimly lit room without talking, reading, eating, or listening to music between injection of FDG tracer and PET/CT imaging. PET images were acquired using 3D acquisition mode, with corrections for decay, random radiation, and scatter, using 10 minute bed positions. Images were reconstructed into 47 axial slices per bed position, composed of isotropic voxels of 2.23 mm, using iterative reconstruction with 21 subsets. CT images were acquired using mA of 215, 120 kV, a pitch of 1.75:1, rotation speed of 0.5 sec., with beam collimation of 10 mm using 16 detectors of 0.625 mm, and a table speed of 17.5 mm per rotation. All PET images were corrected for attenuation using the CT images which are intrinsically maps of tissue attenuation. PET and CT images were visually evaluated for proper anatomic co-registration, and if needed, were corrected for any potential mis-registration using ACQC co-registration software, with repeat PET reconstruction after corrected co-registration. Images were reviewed on a Xeleris workstation with additional analysis and review using iSSP35 software (University of Washington) for quantitative database comparisons for identification of areas of relative cortical hypometabolism or hypermetabolism, using global brain normalization.

Motor cortex placement was determined by using standard anatomical landmarks as seen in the CT scans. The latter was confirmed with rTMS motor activation.

For MRI data acquisition, imaging was performed at MICSC on a 3T Siemens Verio (Erlangen, Germany). The protocol consisted of acquiring structural images followed by functional ones. Structural images were acquired with a 3D Magnetization Prepared Rapid Gradient Echo (MPRAGE) pulse sequence (TR/TE/TI=2100/2.74/1100 ms, FA=12, 176 sagittal slices, resolution=1.0×1.0×1.0 mm³). Resting State and GIST datasets were acquired with a gradient echo Echo Planar imaging Sequence (EPI) with TR/TE=2.5 s/30 ms, 38 slices (thickness of 3.5 mm, in-plane resolution of 3.5 mm and a matrix size of 64×64), a total of 126 volumes were acquired. The paradigm for performing GIST is described below.

For data analysis, functional imaging data (RSN and GIST) was analyzed using fsl (http://www.fmrib.ox.ac.uk) tools. Structural images were tripped the skull for alignment with functional images. RSN and GIST were analyzed with FSL's melodic tool, a statistical tool to perform independent component analysis (ICA). Images were subject to the following pre-processing steps: motion corrected spatial smoothing (5 mm), and high-pass temporal filtering (100 s cut off). The resulting statistical maps of the ICA analysis were inspected for their anatomical patterns to determine those that encompassed structures of interest (DLPFC).

Block design protocols were used to evaluate BOLD fMRI responses to finger tapping for sensorimotor activation and to word generation for language network activation. For active conditions of the block design for finger tapping, the patient was shown an imaging of a hand on an LCD screen (ESys and Dynasuite from Invivo Corporation, Gainesville, Fla.). For the active phase of the word generation task, the patient was given directions in advance to think of many words as they could that started with the letter shown for fifteen seconds of each active period (ESys and Dynasuite from Invivo Corporation, Gainesville, Fla.). The motorstrip was identified with the fMRI data set by identifying the area of activation with contra lateral finger tapping and confirmed by rTMS motor activation. MRI data acquisition was performed with a 2D Echo Planar sequence with a TR of 2 seconds, a TE of 30 mseconds, a slice thickness of 4 mm and a matrix of 64×64.

For both PET/CT and MRI data sets, the Brainlab device was used to target the dorsal lateral prefrontal treatment site by finding a location that was at least five centimeters anterior to the sensorimotor area and coincident with the superior frontal sulcus (junction of superior and middle gyrus). The language activation scans activated middle and lower portions of the prefrontal region serving as a confirmation of areas forming a ventral non targeting zone. In addition, the area with lowest counts in the PET data set in this region was selected. With fMRI data, the dorsal lateral frontal target was modified to overlap the area of activation seen by the executive network on independent component analysis of resting BOLD and state dependent BOLD that was obtained during a cognitive task, the Guided Imaging Stimulation Test (GIST).

The GIST was used as a stimulus for the state dependent BOLD fMRI and was also used as the cognitive paradigm for stimulation during rTMS treatment sessions. GIST was designed by having the patient generate thirty words that represented people, places or activities that each subject would like to have experiences with once the depression is treated successfully. Three words from the list was then presented visually for thirty seconds; the patient was instructed to choose one of the words and try to image a scene utilizing the word for a duration of thirty seconds. After each thirty second interval another set of three words was presented. The patients were further instructed to choose a new word if any repeats of the three word sets were encountered. Patients were also instructed to click a hand counter when a successful imaging was experienced. In this manner, the patient's performance required decision making, imagination and positive self-projections into the future. In addition, working memory was required for proper task performance because of the instruction for non-repetition of choices. The request to have the patient click on the hand counter also placed a requirement for self-assessment.

Quality assurance protocols required the technician to check the scalp marking with the Brainlab target set on a weekly basis and whenever the permanent ink marking may have been obscured. The concordance of technician generated targeting with physician generated targeting was checked on two occasions for all of the imaging guided patients. Any discrepancy of greater than one cm was logged as a quality assurance failure.

The primary measure was a good outcome as determined by a more than fifty percent decrease in Beck Depression scores as well as the patient's sense of marked improvement in mood with improved ability to perform daily activities.

Results.

There were 12 patients in the non-navigated group with nine women and 3 men. The average age was 57+/−11 years; ten of them scored in the moderate to severe depression range. All of them had unipolar depression and none had psychotic features. In the navigated group there were 73 patients including 37 women and 36 men with an average age of 50+/−17 years. Fifty seven of the 73 were in the moderate to severe range of depression scores. Of the navigated group, five had bipolar mood disorder by history and ten had psychotic features including hallucinations, ideas of reference or other delusions. There were no significant differences between groups with regards to age, depression severity, number of medications or numbers of patients who were medication free (see table 1).

In the imaging navigated group, the Brainlab device with fMRI based imaging enabled the technician to localize the scalp site of low threshold contralateral hand activation very rapidly within 2 to 5 minutes on each occasion. The majority of instances required little or no adjustment and all of the final localizations were within one cm of the sites determined by Brainlab in the planning stages before the patient was co-registered to the virtual cranium. Motor strip localization without imaging guidance required 5 to 35 minutes. Quality checks within the sessions for the imaging guided patients with Brainlab localization yielded 5 instances out of 146 measurements of inter observer disagreement of one cm or more. These results contrast with 12 out of 24 instances of quality assessment failures using the standard Neuronetics device and protocol. Cranial tilting away from the magnetic coil with ineffective counter pressure from the head holder armature appeared to be an important source of variability with the standard targeting device.

Debriefing of patients after sessions in phase one was remarkable for repeated experiences of brief napping interrupted by the technician's admonitions, “day dreaming” and various other cognitive states. In phase two, patients were able to successfully imagine pleasance scenes guided by GIST throughout the treatment sessions as detected by event recorder totals and debriefing.

Remission was obtained in the imaging navigated group of phase two, in 48 out of 73 patients (66%) compared to 3 out of 12 patients (25%) in the phase one, non-navigated group (p=0.0109, two tailed Exact). For patients with follow up information of at least 6 months, the duration of remission was 12.5+/−12.0 months for imaging guidance patients and 4.0+/−3.6 months for non-imaging guided patients. None of the non-imaging navigated patients responded for more than one year compared with 18 out of 39 in the navigated group (p=0.0041 two tailed Exact).

In seven patients treated with left DLPF magnetic stimulation, PET scans demonstrated an asymmetrically worse hypometabolism in the right dorsal lateral prefrontal resulting in five remissions; the 71% remission rate is not statistically different in comparison to a 63% remission rate (39/62) when patients with left-sided or bilateral frontal hypometabolism were treated with left DLPF targeting. Only 1 out of 7 (14%) of left handers had remission when treated with left DLPF targeting compared to 44 out of 65 (68%) of right handers (p=0.0012). When this pattern of failure became apparent in the earlier clinical experience, subsequent left handers were offered right DLPF stimulation with a remission rate of 75% (3 out of 4). Four patients in the imaging group also had rTMS to the left temporal region at one Hz for attempted suppression of auditory hallucinations; this was successful in 2 of them.

Conclusions.

In this experience it was possible to show that technically advanced imaging guided neuronavigation can be successfully translated to a clinical setting with subsequent improvements in technical reliability and outcomes. The performance of frameless stereotaxis has been shown in phantoms and in surgical settings to be in the millimeter range of accuracy. The reported experience in the present clinical series is in line with the latter observations.

Improved clinical outcomes were found in the group targeted with imaging guidance. A randomized controlled study would be potentially helpful in further determining the incremental benefit of imaging based navigation over non-imaging guidance targeting protocols. Compared to published studies, outcomes in the non-navigated patients in the present study are similar to previously reported experiences. By contrast, the outcomes for imaging guided rTMS patients appear to be better than those previously reported and better than the non-navigated patient in the present study.

Although the PET scans in the present study were used to target a region of hypometabolism in the DLPF within the targeted hemisphere, the question of redirecting targets based upon which hemisphere is asymmetrically more hypometabolic is not entirely answered by the present experience. The finding of a minor asymmetry on a single PET scan may not be sufficiently predictive of proper targeting lateralization. The application of Arterial Spin Labeling MRI (ASL) would be expected to a more reliable and predictive means of targeting the best site since it can be repeated in a way that may minimize random and state dependent fluctuations in signal. ASL can be repeated on numerous occasions since it does not expose the individual to repeated radiation exposure.

The present experience with imaging guidance confirms earlier suggestions that the five centimeter rule may miss the best targets in many patients. Several factors may have contributed to successful targeting in the present study. Neuronavigation with the patient's own anatomical scans allowed an accurate placement over a sulcus which may allow for more effective interaction of the magnetic field with cortical tissue. The latter would be an advantage compared to navigation based on the standard five centimeter rule. In addition, targeting with functional data from PET and fMRI may allow for a better stimulation of those neural networks which may be relevant for achieving remission.

fMRI targeting has advantages over PET scanning in the avoidance of radiation exposure, particularly if repeated scanning is required. fMRI using standard block design protocols can show a robust BOLD effect in the sensorimotor area; localization of the motor strip obtained in this manner facilitates targeting the magnetic coils in the determinations of motor thresholds at the inception of treatment that are required for the determination of the magnetic intensity levels needed for effective cortical activation.

The present clinical experience also points out the poor outcomes encountered when left handers are treated in the same manner as right handers. Protocol revisions need to be considered for left handed individuals.

Besides demonstrating the increased precision afforded with imaging based neuronavigation, the present study utilized a cognitive task concurrent with rTMS which may have added to improved outcomes. The task not only keeps patients awake and busy; a simple analysis would suggest that the task may be “pre-stimulating” the dorsal lateral prefrontal area because of this area's involvement in imagination, working memory, self-assessment and decision making. As the patient is asked to imagine themselves performing competently and happily in the imagery tasks, this approach seems to many cognitive behavioral techniques with rTMS.

### REFERENCES

1. O Gonzalez, J T Berry, L R McKnight-Eily, T Strine., V J Edwards, H Lu, M S J B Croft, PhD. (2010): Current Depression Among Adults-United States, 2006 and 2008. Morbidity and Mortality Weekly Reports 59; 1229-1235.
2. John P. O'Reardon, H. Brent Solvason, Philip G. Janicak, Shirlene Sampson, Keith E. Isenberg, Ziad Nahas, William M. McDonald, David Avery, Paul B. Fitzgerald, Colleen Loo, Mark A. Demitrack, Mark S. George, and Harold A. Sackeim (2007): Efficacy and Safety of Transcranial Magnetic Stimulation in the Acute Treatment of Major Depression: A Multisite Randomized
3. Tal Herbsman, David Avery, Dave Ramsey, Paul Holtzheimer, Chandra Wadjik, Frances Hardaway, David Haynor, Mark S. George, and Ziad Nahas. (2009): More Lateral and Anterior Prefrontal Coil Location Is Associated with Better Repetitive Transcranial Magnetic Stimulation Antidepressant Response. Biological Psychiatry 66: 509-515.
4. Kengo Shimoda and Robert G. Robinson. (1999): The Relationship between Poststroke Depression and Lesion Location in Long-Term Follow-up. Biological Psychiatry 45: 187-192.
5. Francoise Biver, Serge Goldman, Veronique Delvenne, Andre Luxen, Viviane De Maertelaer, Philippe Hubain, Julien Mendlewicz, Francoise Lotstra. Frontal and parietal metabolic disturbances in unipolar depression Biological Psychiatry Volume 36, Issue 6, Pages 381-388, 15 Sep. 1994
6. Christopher J. Bench, Karl J. Friston, Richard G. Brown, Lynette C. Scott, Richard S. J. Frackowiak and Raymond J. Dolan. (1992): The anatomy of melancholia-focal abnormalities of cerebral blood flow in major depression. 22:607-615
7. M Petrides and D N Pandya. (1999): Dorsolateral prefrontal cortex: comparative cytoarchitectonic analysis in the human and the macaque brain and corticocortical connection patterns. European Journal of Neuroscience 11: 1011-1036.
8. Marie-Laure Paillere Martinot, Andre Galinowski, Damien Ringuenet, Thierry Gallarda Jean-Pascal Lefaucheur, Frank Bellivier, Christine Picq, Pascale Bruguiere, Jean-Francois Mangin, Denis Riviere, Jean-Claude Willer, Bruno Falissard, Marion Leboyer, Jean-Pierre Olie, Eric Artiges and Jean-Luc Martino. (2010): Influence of prefrontal target region on the efficacy of repetitive transcranial magnetic stimulation in patients with medication-resistant depression: a [¹⁸F]-fluorodeoxyglucose PET and MRI study. International Journal of Neuropsychopharmacology 13:45-59.
9. Uwe Herwig, Yvonne Lampe, Freimut D. Juengling, Arthur Wunderlich, Henrik Walter, Manfred Spitzer, Carlos Schonfeldt-Lecuona. (2003): Add-on rTMS for treatment of depression: a pilot study using stereotaxic coil-navigation according to PET data. Journal of Psychiatric Research 37: 267-275.
10. Mauro Garcia-Toro, Joan Salva, Jaume Daumal, Joana Andres, Maria Romera, Oriol Lafau, Miguel Echevarria, Martin Mestre, Carmen Bosch, Catiana Collado, Olga Ibarra, Iratxe Aguirre. (2006): High (20-Hz) and low (1-Hz) frequency transcranial magnetic stimulation as adjuvant treatment in medication-resistant depression. Psychiatric Research: Neuroimaging 146: 53-57.
11. Michael D. Fox, Mark A. Halko, Mark C. Eldaief, Alvaro Pascual-Leone. (2012): Measuring and manipulating brain connectivity with resting state functional connectivity magnetic resonance imaging (fcMRI) and transcranial magnetic stimulation (TMS). NeuroImage Article in Press.
12. Wassermann, E. M., Wang, B., Zeffiro, T. A. Sadato, N., Pascual-Leone, A., Toro, C. et al 1996 Locating the motor cortex on the MRI with transcranial magnetic stimulation and PET. NeuroImage 3, 1-9.
13. Alvaro Pascual-Leone, David Bartres-Fazt and Julian P. Keenan Transcranial magnetic stimulation: studying the brain-behavior relationship by induction of ‘virtual lesions’. Phil Trans R Soc Lond B (1999) 354, 1229-1238.

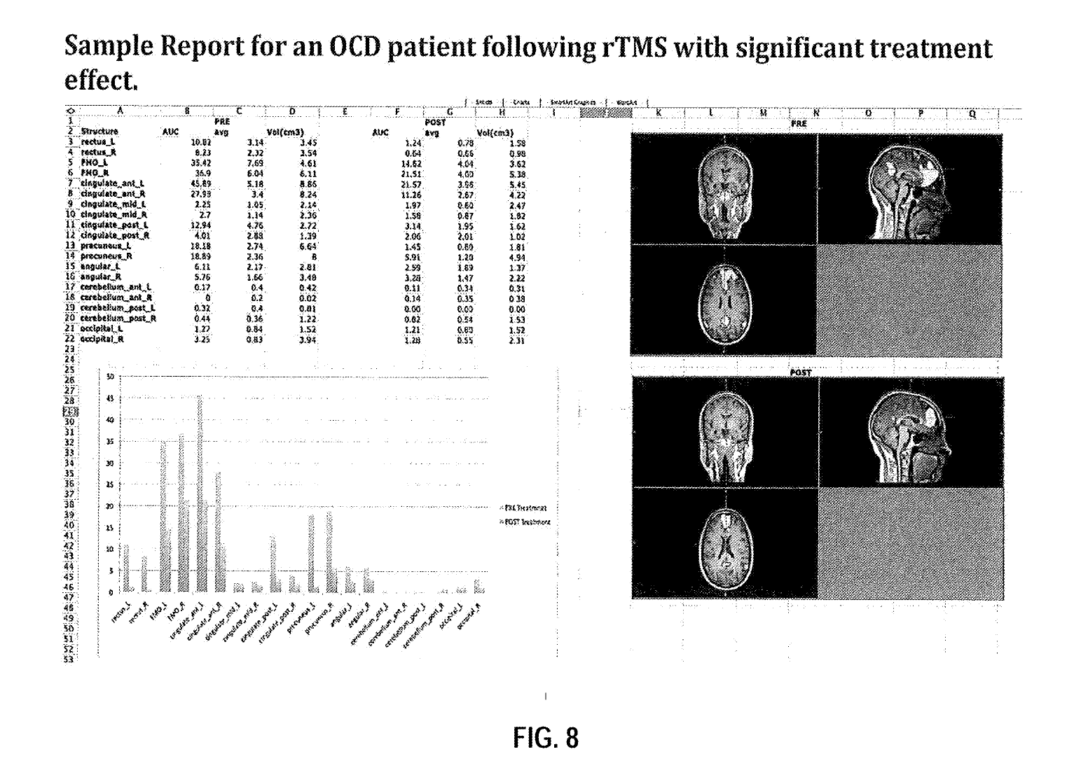

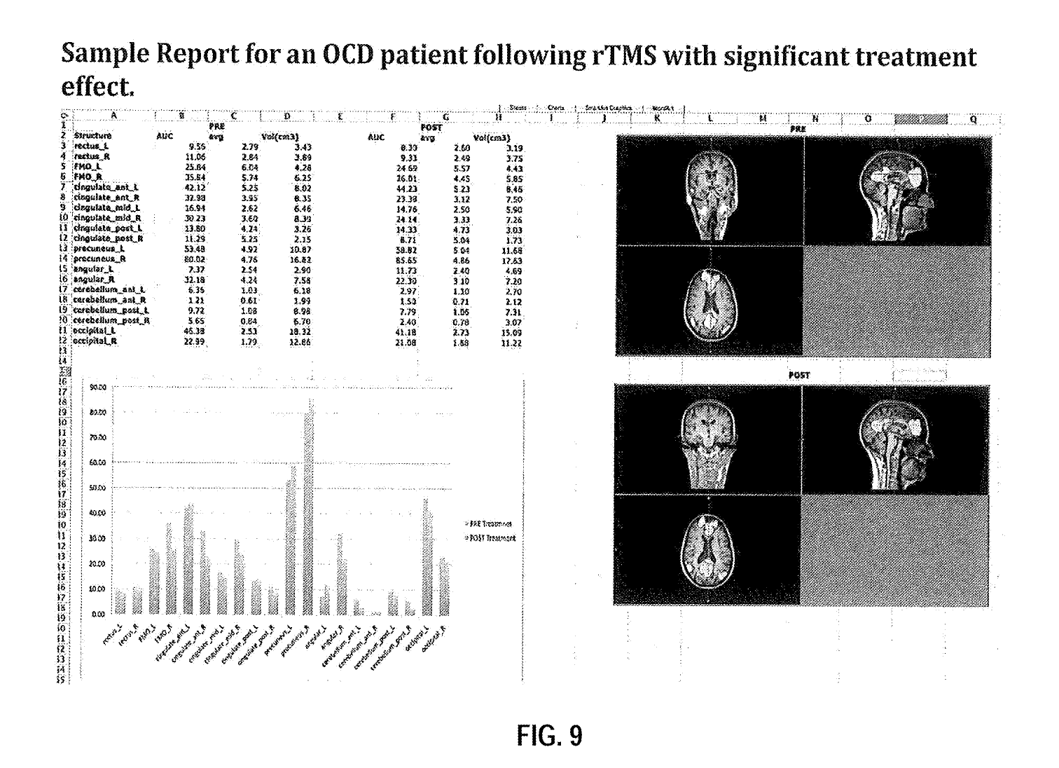

FIGS. 8 and 9 provide examples of application of methods to determine effectiveness of imaging-guided rTMS of OCD patients. Patients have been taken for treatment of refractory OCD with rTMS. As described herein, a series of fMRI scans are acquired and analyzed to determine the brain area to be targeted by rTMS. Following treatment, patients undergo a similar series of fMRI scans to assess treatment effects. FIGS. 8 and 9 present two examples. FIG. 8 displays information that is generated by the methods of this application in one patient that had a significant treatment effect with a reduction of the standard clinical measure (Y-BOCS) of more than 50%. FIG. 8 displays three views of the brain network pre- (top right panel) and post- (bottom right panel) treatment. Visual inspection indicates a reduction of yellow/red areas. The Top left panel quantifies those changes numerically and are displayed as a bar graph (bottom left panel). For the bar graph; blue bars indicate level of connectivity in different brain structures in the network known to be altered in OCD and red bars indicate connectivity in the same brain structures post treatment. These changes will have a statistical significance measure when they are compared against a database of normal subjects.

FIG. 9 indicates similar information for a patients whose clinical Y-BOCS scores remained unchanged following treatment. Visual inspection of brain activity quickly indicate a lack of treatment effect. Quantitative analysis further corroborates that initial assessment.

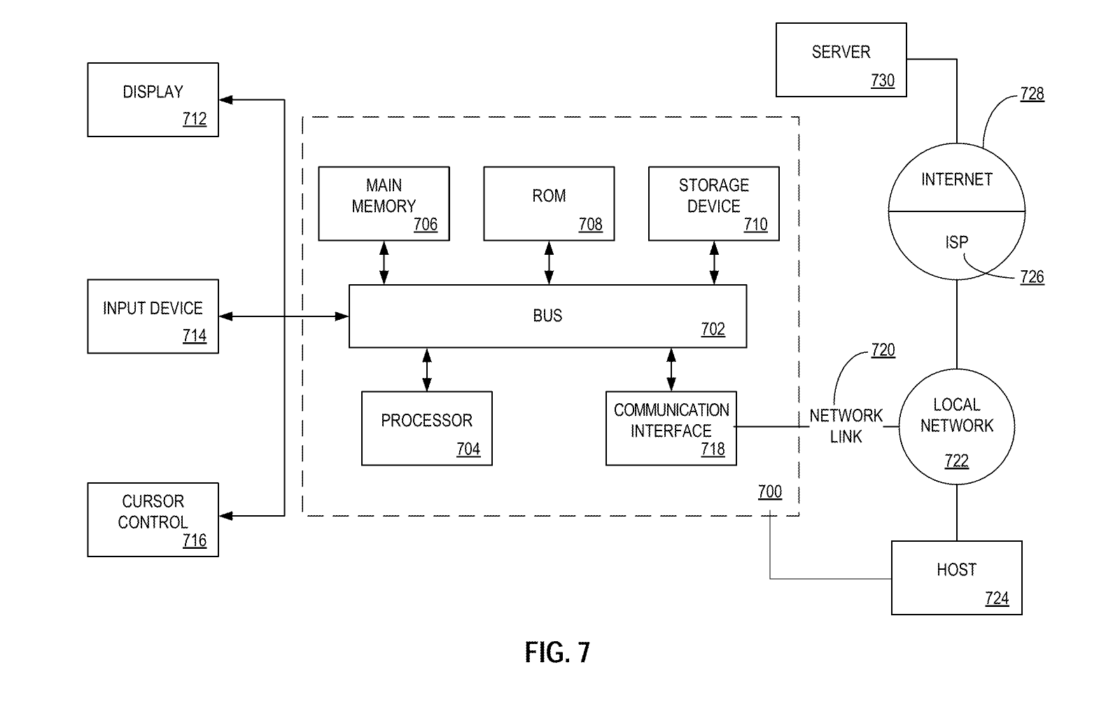

FIG. 7 is a block diagram that illustrates a computer system 700 upon which some embodiments may be implemented. Computer system 700 includes a bus 702 or other communication mechanism for communicating information, and a processor 704 coupled with bus 702 for processing information. Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Computer system 700 further includes a read only memory (ROM) 708 or other static storage device coupled to bus 702 for storing static information and instructions for processor 704. A storage device 710, such as a magnetic disk, optical disk, or a flash memory device, is provided and coupled to bus 702 for storing information and instructions.

Computer system 700 may be coupled via bus 702 to a display 712, such as a cathode ray tube (CRT) or liquid crystal display (LCD), for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane. In some embodiments, input device 714 is integrated into display 712, such as a touchscreen display for communication command selection to processor 704. Another type of input device includes a video camera, a depth camera, or a 3D camera. Another type of input device includes a voice command input device, such as a microphone operatively coupled to speech interpretation module for communication command selection to processor 704.

Some embodiments are related to the use of computer system 700 for implementing the techniques described herein. According to some embodiments, those techniques are performed by computer system 700 in response to processor 704 executing one or more sequences of one or more instructions contained in main memory 706. Such instructions may be read into main memory 706 from another machine-readable medium, such as storage device 710. Execution of the sequences of instructions contained in main memory 706 causes processor 704 to perform the process steps described herein. In alternative embodiments, hard-wired circuitry may be used in place of or in combination with software instructions to implement the invention. Thus, embodiments are not limited to any specific combination of hardware circuitry and software. In further embodiments, multiple computer systems 700 are operatively coupled to implement the embodiments in a distributed system.

The terms “machine-readable medium” as used herein refer to any medium that participates in providing data that causes a machine to operate in a specific fashion. In an embodiment implemented using computer system 700, various machine-readable media are involved, for example, in providing instructions to processor 704 for execution. Such a medium may take many forms, including but not limited to storage media and transmission media. Storage media includes both non-volatile media and volatile media. Non-volatile media includes, for example, optical disks, magnetic disks, or flash memory devices, such as storage device 710. Volatile media includes dynamic memory, such as main memory 706. Transmission media includes coaxial cables, copper wire and fiber optics, including the wires that comprise bus 702. Transmission media can also take the form of acoustic or light waves, such as those generated during radio-wave and infra-red data communications. All such media must be tangible to enable the instructions carried by the media to be detected by a physical mechanism that reads the instructions into a machine.

Common forms of machine-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, and EPROM, a FLASH-EPROM, flash memory device, any other memory chip or cartridge, a carrier wave as described hereinafter, or any other medium from which a computer can read.

Various forms of machine-readable media may be involved in carrying one or more sequences of one or more instructions to processor 704 for execution. For example, the instructions may initially be carried on a magnetic disk of a remote computer. The remote computer can load the instructions into its dynamic memory and send the instructions over a data transmission line using a modem. A modem local to computer system 700 can receive the data on the data transmission line and use an infra-red transmitter to convert the data to an infra-red signal. An infra-red detector can receive the data carried in the infra-red signal and appropriate circuitry can place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card or other internet connection device, or a modem to provide a data communication connection to a corresponding type of data transmission line. As another example, communication interface 718 may be a local area network (LAN) card to provide a data communication connection to a compatible LAN. Wireless network links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the Internet 728. Local network 722 and Internet 728 both use electrical, electromagnetic or optical signals that carry digital data streams. The signals through the various networks and the signals on network link 720 and through communication interface 718, which carry the digital data to and from computer system 700, are exemplary forms of carrier waves transporting the information.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718.

The received code may be executed by processor 704 as it is received, and/or stored in storage device 710, or other non-volatile storage for later execution. In this manner, computer system 700 may obtain application code in the form of a carrier wave.

Other features, aspects and objects of the invention can be obtained from a review of the figures and the claims. It is to be understood that other embodiments of the invention can be developed and fall within the spirit and scope of the invention and claims.

The foregoing description of preferred embodiments of the present invention has been provided for the purposes of illustration and description. It is not intended to be exhaustive or to limit the invention to the precise forms disclosed. Various additions, deletions and modifications are contemplated as being within its scope. The scope of the invention is, therefore, indicated by the appended claims rather than the foregoing description. Further, all changes which may fall within the meaning and range of equivalency of the claims and elements and features thereof are to be embraced within their scope.

What is claimed is:

1. A method of measuring the brain activity of a patient with OCD comprising the steps of:
(a) introducing the patient into an MRI machine;
(b) while the MRI machine is running, taking baseline measurements of the patient and subjecting the patient to various stress-inducing tests, such as the elevated arm stress test or the leg elevated stress test, and
(c) evaluating the results.
